(19) United States
(12) Patent Application Publication
(10) Pub. No.: US 2019/0038569 A1
(43) Pub. Date: Feb. 7, 2019

(54) Multifunctional-reinforced dressing

(71) Applicant: Carlos A. Alvarado, Guatemala (GT)

(72) Inventor: Carlos A. Alvarado, Guatemala (GT)

(21) Appl. No.: 15/998,293

(22) PCT Filed: Aug. 1, 2016
(86) PCT No.: PCT/IB2016/054605

§ 371 Date: Aug. 2, 2018

(51) Int. Cl. A61K 9/70; A61K 9/00; A61K 35/644; A61L 15/24; A61L 15/42; A61L 15/44; A61K 36/48; A61K 31/185; A61L 15/28

(52) U.S. Cl. CPC A61K 9/7007; A61K 9/0014; A61K 35/644; A61L 15/24; A61L 15/42; A61L 15/44; A61L 2300/418; A61K 31/185; A61L 15/28; A61L 2300/412; A61L 2300/30; A61L 2300/404; A61K 36/48

(57) ABSTRACT

This invention relates to a surgical or medical dressing which comprises a supporting and reinforcing layer of elastomeric material (as polyethylene homopolymer) embedded in the composition of honey/additives, to supplement the mechanical strength, which would be fabricated as a single integral piece, intended to provide multi-functional properties in the wound environment, particularly for promote or expedite the regeneration or repair deep tissue injuries or chronic wounds such as ulcers.

Several additional characteristics of this new dressing provide an advantage to any wound care product developed from it. First, the biomaterial retains many biological properties of its major constituents (honey/additives). Second, the application of this safety dressing is greatly simplified particularly for pediatric burn Care.

### FIELD OF THE INVENTION

This invention relates to surgical or medical dressing, with a supporting and reinforcing layer of elastomeric material embedded in the composition of honey/additives, suitable for promote or expedite the regeneration or repair deep tissue injuries or chronic wounds such as ulcers, specially designed for assisting the application or placement on the wound site, and relates to a process for the production of such a dressing.

### BACKGROUND AND SUMMARY OF THE INVENTION

During the past two decades, the author has focused his research on advanced treatment therapies for acute and chronic wounds, and the actions associated with them, in the context of wound healing.

The present invention closely conforms to the progress of a previously studied dressing, referring to the International Patent Publications No. WO 2014041440 A1 Publication date Mar. 20, 2014 earlier prepared by the inventor, whose variations give rise to a new invention.

The product described in this patent, is a multifunctional-type dressing, that has superb healing properties, for many patients, especially those with second degree burns, and chronic wounds that are difficult to heal, due to skin and soft tissue infections associated with severe tissue necrosis.

Previous studies demonstrated successful application of this multifunctional-type dressings, for infected wounds or at risk of infection, partial thickness burns, leg, diabetic, and pressure ulcers, traumatic wounds, donor and recipient graft sites.

The dressing can act as a physical barrier to protect the wound until the continuity of the skin is restored, keeping it clean, and preventing bacterial contamination from the external environment.

Relevant aspect of this dressing is the pH reduction, which is obtained by three products in the formulation of the previous art: honey, methyl-Phenol [Policresulen (Condensation product of metacresolsulfonic acid & methanal)], and acetic acid.

According to the reference by Nagoba et al¹, and Schneider², the pH value within the wound milieu directly and indirectly influences all biochemical reactions taking place in the process of wound healing.

The reference of Nagobal, extends the value of pH reduction, with the following statement copied literally “It has been proven that the surface pH of a wound plays an important role in wound healing as it helps control infection and increase antimicrobial activity, oxygen release, angiogenesis, protease activity, and bacterial toxicity.”

Therefore, pH value affects the regular cellular events in wound healing. It has also been observed that wounds with a high alkaline pH have a lower healing rate in both acute and chronic wounds as compared to wounds with a pH closer to neutral.

Wound healing progression decreases when pH is elevated to alkaline condition. The environment of acute as well as chronic wounds progresses from an alkaline state to a neutral state and then to an acidic state when healing begins.³⁻⁷

With respect to the honey, many recent studies demonstrates the effectiveness of honey in wound healing and Peter Molan⁸⁻¹⁰ produces a wide range of publications, focusing on various aspects of its practical applications, and also the large number of patents granted, including the following: EP 0909557 A1, US 20030136274, US 20040054313, US 20040121020, US 20040127826, US 20040127826, US 20040127837, US 20040131693, US 20040258765, US 20050033213, US 20050255166, US 20060099166, US 20080125617, US 20090012440, US 20100049262, US 20100233283, US 20140127283, US 20140142522, US 20150080815, US 2015015093, U.S. Pat. No. 3,767,784, U.S. Pat. No. 5,785,972, U.S. Pat. No. 5,980,875, U.S. Pat. No. 6,956,144, U.S. Pat. No. 7,563,224, U.S. Pat. No. 8,303,551, U.S. Pat. No. 8,568,790, U.S. Pat. No. 8,632,810, U.S. Pat. No. 8,815,298, U.S. Pat. No. 9,044,489, U.S. Pat. No. 9,180,219, U.S. D 7,45,690, U.S. RE 42755, WO 1999055349 A1, WO 2001041776 A2, WO 2001041776 A2, WO 2001067888 A1, WO 2002000269 A1, WO 2002030467 A2, WO 2002087644 A1, WO 2003005978 A1, WO 2003047642 A1, WO 2004000339 A1, WO 2005000032 A1, WO 2005077402 A1, WO 2007009185 A1, WO 2007030023 A1, WO 2007048193 A1, WO 2007137369 A1, WO 2008049578 A2, WO 2011046454 A1, WO 2012134770 A1, WO 2015110552 A1, WO 2016007776 A1, WO 2009147402 A3.

Various bioactive/agents have been included in the formula for the author to augment the performance of the honey, including pharmaceutical active agents, and plant extracts of the Fabaceae family (anti-inflammatory properties).

Such bioactive agents are particularly advantageous in clinical use because they have other biochemical, medical and stimulating effects to facilitate tissue repair and/or regeneration.

A major advance in the use of this product, is the evidence that few dressing changes are needed during the period of treatment required, to achieve the optimum healing.

In our understanding, the most significant factors that affect wound healing, are mainly due to repetitive dressing changes and multiple interventions in burn-wound site.

A clear link was demonstrated in all our studies: repetitive dressing changes and multiple interventions increase the risk of hypertrophic scarring.

To reduce these problems our approach for wound-burn care has been oriented to establish the best management strategies for each patient, and from there the special recognition to obviate the repetitive redressing of the burn-wound area.

The author thinks this is the key in terms of being able to modify the healing response and reduce the severity of subsequent scarring.

Dowsett¹¹ reported that, “frequent dressing changes are often inconvenient for the patient and costly for the health services in terms of clinician time and dressings product usage”.

It is necessary emphasize that children, especially those between 0 and 4 years old, have a thin skin, and the physical protection of the wound against mechanical disturbances play a significant role in an undisturbed wound healing.

Fritz Patent WO 2009105761 A2 Aug. 27, 2009 made the following observation: “Many dressings can adhere to the surface of delicate de novo epidermal layer and can result in extensive tissue scarring when frequent changes in dressings may be required. Insufficient wound care can significantly reduce the healing rate, promote other secondary complications such as infections, and induce additional discomfort and pain”.

Additionally, Meaume et al¹², refer that it is important to remember that for many patients the removal and/or changing of a dressing is the most common trigger for pain.

Frequent changes of dressings on such wounds tend to interfere with the healing of the wounds, and such frequent changes may also increase the possibility that the wound can be contaminated by bacteria transmitted from the nurse or other specialist who is changing the dressing.

Therefore, in the pediatrics population, a dressing that requires infrequent reapplication has obvious benefits by decreasing the number of dressing change procedure the child must undergo.

If necessary, preferably made in a gentle, no painful, and in an atraumatic manner, to effectively prevent or minimize disturbance to the tissues.

Regarding the previous patent by the inventor, unfortunately, there are several practical problems to be overcome, one of which refer to the fragility of the dressing, because is prone to break into fragments dissociated from the primary composition i.e. mechanically weak, and insufficient stability.

Other concern to the author, is dressing-related difficulties in the treatment of extensive injuries, such as bums, and extensive leg ulcers, since may be impractical and time-consuming for the healthcare professional.

Reference is made to the disposition of the material in individual unconnected sections, which makes technically laborious the coverage of large surfaces, that interfere the rapid application.

Other additional problem associated with these complicated applications becomes apparent when one considers that the human body essentially has no flat surfaces; it is comprised of many complex curves, and if the product is fragmented, the application is intricate.

Another practical problem that has been considered, means that this product has been used in inactive persons, without considering the extremely mobility of children, who need more freedom of movement than an adult.

As related by Dorsett-Martin and Col¹³., considerations for dressings specifically designed for pediatric patients include achieving adherence of dressings due to ever changing shear forces produced during play behavior of naturally active toddlers.

This author also described that the evidence-based choices for treating burns in children are not well defined, and inadequate care may cause lifelong damage in their bodies that often cannot be repaired later in life.

There are undoubtedly several patents also referencing the use of various gels and polymers for medical use and in particular for wound dressings, but these materials have not been optimized to satisfy the early freedom of movement required in children.

Large and increasing number of agents are available with varying claims of meeting the problems described previously but most of the original promise has been unfulfilled.

In relation to above problems various products for wound care are known to the art, all, or almost all of them suffer from one or more than one disadvantage.

In relation to the above problems existing products may have varying effectiveness, and some have been shown to cause tissue damage and slowing down the healing process.

In the respective order that the problems in the prior art were presented, solutions to those problems, and advantages of the present innovation are listed.

In the context of fragility of the dressing, it is a common practice in the pharmaceutical and biotechnology industry, to include a support in their devices for many purposes.

For example, the product Biobrane®, a biosynthetic a bilaminar material composed of an outer silicone film with partially embedded nylon and porcine type one collagen incorporated into both components.

It is noted by Rogers and col.¹⁴, that this product presents a significant limitation in relation to the use for superficial partial thickness facial bums in children, particularly in those with black skin, in whom changes in pigmentation on the face can have devastating cosmetic sequelae.

U.S. Pat. No. 5,593,441 to Lichtenstein et al, describes a composite prosthesis preferably having a sheet of polypropylene mesh that allows tissue in-growth, such as Marlex® mesh. This reference discloses that other surgical materials that are suitable for tissue reinforcement and defect closure may be utilized, including absorbable meshes such as polyglactin (Vicryl®) mesh.

Substantial work has also been performed relating to the creation of bioabsorbable and includable, cell growth enhancing matrices, lattices, or scaffolds.

Exemplary patents known to applicant include: U.S. Pat. No. 4,649,909, U.S. Pat. No. 4,664,662 A, EP 0274898, A2 U.S. Pat. No. 4,997,425 A, U.S. Pat. No. 5,086,764, U.S. Pat. No. 5,527,271, U.S. Pat. No. 5,891,558 A, US 20030120229, U.S. Pat. No. 6,605,751, U.S. Pat. No. 6,967,261, U.S. Pat. No. 7,005,556, U.S. Pat. No. 7,220,889, WO 2007149958 A2, US 20080014386, US 20080027366, U.S. Pat. No. 7,714,183, US 20110135726, and U.S. Pat. No. 8,067,662; the disclosures of which are incorporated by reference herein.

Bioactive, bioabsorbable ceramic fillers and fibers and/or their use in bioabsorbable devices as bioactive ceramic fillers and/or reinforcements have been described in several of the publications, and also are describe in, e.g., EPO Pat. Appl. 0 146 398, U.S. Pat. No. 4,612,923, and PCT Pat. Appl. WO 96/21628, the entire disclosures of each of which are incorporated herein by way of this reference.

The World Union of Wound Healing Societies (WUWHS)¹⁵ has recommended that dressings ensuring several practical features, including the following. “The dressing should: Stay intact and remain in place throughout wear time, Achieve the desired moisture level, prevent leakage between dressing changes, not cause maceration, allergy or sensitivity, be comfortable, conformable and not impede physical activity, be suitable for leaving in place for a long time.”

Dressings should be as easy as possible for a user to apply to a wound site. Users typically attempt to apply a dressing to a wound as quickly as possible to bring a healing-promoting environment to the wound speedily.

Despite the availability of a variety of wound dressings, there is a need and a demand for an improved wound dressing which prevents trauma upon wound dressing changes, improves the durability and lifetime of the dressing, anatomically conforms to a wound and possesses improved application and removal properties.

According to the present invention there is provided a medical-surgical dressing comprises a supporting and reinforcing layer of reticulated elastomeric material (as polyethylene polymer), embedded and connected entirely in the honey/additives composition, to provide security against disintegration. This ensures that do not fragment under normal handling conditions.

This provides additional advantages over the physically prior art honey-based versions, because offers structural and functional characteristics, that stabilizes the dressing to prevent fragmentation, and additionally produce flexibility of the finished products.

For over forty years, the various products that the author has investigated, are associated as a secondary dressing for fixing/maintenance a polymeric material, which has provided surprising results, to effectively assist in the fixation and retention of primary dressing, even in situations with local infections.

And in that sense the use of such elastomeric materials on the middle of the Honey/additives composition, allow bonding with sutures the individual pieces so that the user covers a total area of bums faster, and achieving a new functional capability from its predecessors.

A better understanding of the influence of these factors on repair, have made our efforts are directed to reducing dressing change frequency, on preventing bum-wound progression and improving patient comfort.

The concept of treating bum-wounds with products that may promote healing and decrease wound conversion, may lead to therapeutics that will promote proper tissue repair and improve impaired wound healing.

Accordingly, the present invention possesses numerous advantages over wound dressings previously known. The present multifunctional wound dressing requires less frequent dressing changes. This results in significant cost savings and enhances the wound healing processes by minimizing disturbances of the wound.

The reinforced tissue dressing of the present invention, provides simplified application procedures, eliminating the prior art difficulties encountered with the unreinforced dressing.

And the construction with the perforated elastomeric material allows connection by interlacing in a series of connected dressings, to extend material for the complete cover of tissues.

The application is simplified by increased size of individual dressing during handling, and application and is easily performed by a single surgeon.

If the application can be simplified, and with the easily handled and rapidly application of the dressing, is possible to significantly reduce the time required of the surgical procedure.

Thus, the dressing can be adapted for application to a plurality of wound types and may be sufficiently conformable to be positioned in direct contact with an irregularly shaped surface of a wound bed.

In publication made by Lawton¹⁶, make the following recommendations: “As epithelialization begins and there is re-establishment of an intact epithelium, the new areas of skin cover are particularly delicate and sensitive to damage. It is important at this stage of healing to take appropriate precautions to prevent damage to the newly restored skin tissue.”

Regarding the mobility of children, our approach has been directed to create the best solution for the patients, using a new approach in areas where there has been no improvement and the emphasis is on the treatment processes, specifically that favoring early child's mobility, and further without damaging the fragile tissues of this group of pediatric population.

There are also other associated difficulties, such as problems with wound exudate, and infection difficulties associated with competition dressings, which the present invention also seeks to address as part of its solutions for addressing these problems.

The present invention overcomes all the prior art difficulties by attaining a totally new, unique membrane formulation.

Where the membranes incorporate a perforated reinforcing material, the overall strength of the membranes is increased, together with their conformability. This latter feature allows the membranes to be molded to fit the surface to be covered.

The true value of this product, rest with the solution offered to people, particularly for pediatric patients. In using this dressing, the patient gains unparalleled, immediate and lasting therapeutic benefits.

### SUMMARY

The invention is directed to a medical-surgical reinforced dressing for promote or expedite the regeneration or repair of skin tissues, which comprises a supporting and reinforcing layer of reticulated elastomeric material (as polyethylene polymer), embedded and connected entirely in the honey/additives composition.

Accordingly, it is an object of this invention to provide a process and method for producing dressings with a suitable reinforced material as a part of the dressing.

It is therefore an object of the present invention provide a dressing which is strong, flexible, which comprises a removable reticulated high-density polyethylene, having a thickness from about 0.8 mils to about 1.0 mils, that has already been proven to be nontoxic and with a well-known record of biological acceptance.

It is another object of the present invention provide wound dressing that easily conform to the shape of the injury and allow optimal contact with the wound area.

Accordingly, it is another object of the present invention to provide simplified application procedures in the wound site, for more rapid fixation, without the undesirable fragmentation in the prior art composition.

It is another object of the present invention provide a method which allows connection of multiple pieces, so that when used, may coincide it to the size of the area of tissue damage.

Still another object of the present invention, is to provide methods and compositions for providing active agents with antimicrobial activity to wound sites and damaged tissues.

It is another object of the present invention provide a wound dressing that in addition to their mechanical effect, provide other biochemical, medical and stimulating effects to facilitate tissue repair and/or regeneration.

It is another object of the present invention provide a wound dressing for speed up the healing process with optimum microenvironment that promotes repair and/or regeneration of the affected tissues, and to provide the removal or replacement thereof with minimal trauma to the wound site.

Another object of the present invention is to provide a wound dressing, the use of which guarantees freedom of early mobility, particularly for children, during the care of their bum-wounds.

### DETAILED DESCRIPTION

In the discussions herein, several terms are used. To provide a clear and consistent understanding of the specification and claims, the following definitions are provided.

Acute—sharp, severe, having rapid onset, severe symptoms and a short course, not chronic.

Angiogenesis—This occurs during the proliferative phase of healing when new blood vessels infiltrate the wound and endothelial budding forms capillaries.

Adhering dressing—the quality of clinging or being closely attached

Antimicrobial agent—preventing the development or pathogenic action of microbes, helps decrease an infection and allows healing of a wound

Bacterial—unicellular microorganisms, lacking chlorophyll, many kinds of bacteria, creating an infection

Chronic—long, drawn out, applied to a disease that is not acute.

Chronic wound—covered with necrotic (dead) material and surrounded by cellulites, also is described as an infected wound.

Debridement—the process of removing dead/unhealthy and/or contaminated tissues, foreign bodies, and other debris on the wound, using mechanical, enzymatic or sharp techniques.

Dressing—Any of various materials utilized for covering and protecting a wound.

Epidermis—the outer layer of skin.

Epithelialization—The process of the formation of new epithelial tissue -the upper layer of the skin.

Eschar—a slough, especially one following a cauterization or burn—any agent used to destroy dead tissue and to cause sloughing which produces an eschar (a blackened area) in the treatment of skin disease.

Excision—Is a surgical procedure requiring incision through the deep dermis (including subcutaneous and deeper tissues) of open wounds, burn eschar, or burn scars.

Exudates—the material that often forms over an open wound. It consists of proteinaceous material from the wound itself.

Granulation tissue—Newly formed vascular tissue (fragile capillaries) normally produced in the healing of wounds of soft tissue and ultimately forming the scar; it consists of small, translucent, pink to red, nodular masses of granulations that fill in an open wound bed during the proliferative phase of healing.

Infection—The host response to bacterial, viral or similar invasion.

Inflammation—A non-specific host response to invasion of foreign material.

Lesion—any visible, local abnormality of the tissues of the body, as a wound, sore, rash, or a boil.

Necrosis—localized tissue death that occurs in groups of cells in response to disease or injury.

Purulent—producing or containing pus.

Sterilization—A process that kills and/or removes all classes of microorganisms and spores.

Stroma—The supportive framework of an organ usually composed of connective tissue.

Superficial—confined to the surface.

Tissue regeneration−Healing in which lost tissue is replaced by proliferation of cells which reconstruct the normal architecture.

Tissue repair—Healing in which lost tissue is replaced by a fibrous scar, which is produced from granulation tissue.

Treatment or treating—refers to any means of control of a condition or disorder, including prevention or prophylaxis, cure and relief, or relief of development of the condition or disorder.

Ulcer—a circumscribed, craterlike lesion of the skin or mucous membrane resulting from necrosis that accompanies some inflammatory, infectious, or malignant processes.

Wound dressing—is a material applied to a wound or a diseased part of the body, with or without medication, to protect and assist healing.

From a wound care perspective, the growing prevalence and incidence of non-healing acute and chronic wounds is a concern, and numerous resources are available to clinicians across practice settings in wound-burn care, with limited success.

Accordingly, it is a general object of this invention to provide medical surgical dressing and methods of use and manufacture, which overcome the disadvantages of the prior art, and which fulfill the need set forth above.

In a first embodiment, the use of appropriate wound care interventions, can substantially improve clinical outcomes and reduce unnecessary morbidity and mortality.

In a specific embodiment, the new technology refers to further improvements in patient care and better procedures involved in wound dressings and surgical wound care.

In a second embodiment, the present disclosure, is directed to a new multi-functional composition of honey/additives, with an internal reinforcing material to supplement the mechanical strength, and for promote or expedite the regeneration or repair of skin tissues.

In another embodiment, there is provided a method of manufacturing a dressing, said method including the steps of: a) preparing a composition including honey/additives in a preferred form; and b) combining said honey composition with a preferred elastomeric material, before gelling of the composition has initiated.

The novel features which are believed to be specific to the invention, will be better understood from the following description of preferred embodiments.

In a specific embodiment, the present disclosure is directed to a new and effective method for promote or expedite the regeneration or repair of skin tissues, with a biocompatible medical-surgical reinforced dressing, which comprises a supporting and reinforcing layer of reticulated elastomeric material (as polyethylene polymer), embedded and connected entirely in the honey/additives composition.

The invention relates, in another embodiment, for the human or veterinary use of novel multifunctional reinforced dressing, to promote or expedite the regeneration or repair of skin tissues, in a variety of injuries or disorders, due to trauma, disease, or surgery.

In the broadest aspect of this embodiment of the invention, the dressing includes a natural honey/additives composition and synthetic polymer of reticulated elastomeric material (as polyethylene polymer), which provides support to the dressing structure.

In a specific embodiment of the invention, the honey/additives composition is reinforced with a biocompatible synthetic polymer.

Continuing the embodiment, the reinforcing dressing preferably comprises a scrim made of reticulated elastomeric material, that has already been proven to be nontoxic and biologically compatible, and polyethylene homopolymer admirably fits this requirement.

In further embodiments of the invention, the inclusion of the reticulated elastomeric material (as polyethylene polymer) in the honey/additives composition, enhance the strength of the product, and thus, provide more security against disintegration of the composition, under normal handling conditions.

Continuing the embodiment, the reinforcing material is preferably in the form of a net, which correspond substantially in shape and dimensions, to those of the dressing to be coextensive therewith.

In this embodiment, the reinforcing material is perforated, to allow bond physically two or more dressings, through the perforations, to the desired shape for its desired uses.

Continuing the embodiment, the incorporation of the elastomeric reinforcing material increases the overall strength of the dressing, together with their conformability, and allows to be molded to fit the surface to be covered.

In another aspect of the invention, the supporting and reinforcing layer of elastomeric material provides structural integrity to provides a desirable combination of tensile strength and conformability, which facilitates the application or removal, or the repositioning of the dressing to the patient's skin.

In another embodiment, the malleability of the dressing improves optimum contact with an irregularly shaped surface of a wound bed, to effect preferred healing.

In various embodiments, the reinforcement elastomeric material provides strength sufficient to permit the user to place and manipulate the dressing onto tissue of a patient, in contemplated applications.

In another embodiment, the dressing of the present disclosure can be used to promote or expedite the regeneration or repair of skin tissues in any subject which require the use, including but not limited to humans, primates, and domestic, farm, pet, or sports animals, such as dogs, horses, cats, sheep, pigs, cattle, rats, mice, and the like.

In another embodiment the present invention provides a wound dressing which offers significant advantages in controlling the environment of the wound surface and which may remain on the surface of the skin over an extended period, until the wound is healed, to the extent that it no longer requires a surgical dressing.

In another embodiment, a further object of this invention is to provide medical dressing which are easy to use, can be readily manufactured, are low in cost, and effective therapeutically for the acceleration of tissue repair.

Continuing the embodiment, the dressing properties allow the use in the medical field as skin protectants, as a general cover for injured or burned skin, as a wound cavity filler, or for exudative, necrotic, dehiscing, and other types of wounds.

In another aspect of the invention, the dressings that may be used to the care of chronic wounds, including non-infected as well as infected wounds, and skin ulcers.

Continuing the embodiment, there is provided a dressing to speed up the healing process, with antimicrobial, anti-inflammatory, and debriding properties.

In certain embodiments, various actives/agents are included to address concerns, that extend beyond the ability of the performance of the honey. These include gelling agents, plant extracts and compounds from the group of phenol and acetic acid.

In a specific embodiment, in the multifunctional reinforced dressing for purposes of the pH reduction and bacterial control, are incorporated three products: raw honey, methyl-Phenol [Policresulen (Condensation product of metacresolsulfonic acid & methanal)], and acetic acid.

Continuing the embodiment, the dressing can be administered by any suitable technique, by removing loose epithelium and necrotic skin tissues at the wound site.

In another embodiment, is provide wound dressings which are easy to apply in very few steps, to fit the shape of the complex curves of a body, and for application to a plurality of wound types.

In various embodiments, the dressing can be maintained in its position on the skin over extended periods of time, which reduces the frequency of dressing changes, and leads to reduced pain, added comfort and mobility to injured children.

In another aspect of the invention, the extended permanence of the dressing over the damaged tissue surface, minimizes disturbances of the wound healing processes, which produces a striking improvement in the recovery of the tissue.

In another embodiment, is attained a reinforced dressing which is capable of being easily handled by a surgeon without the undesirable flaccidity in the prior art composition, leading to ease of application and more rapid fixation,

In one embodiment, the tissue it is cut of a size and shape that is desired for the applicable surgical procedure in which will be used, and if preferred, by interlacing one or more connected dressings by suturing, to cover greater extent of the wounds.

While the compositions and methods of this invention have been described in terms of preferred embodiments, it will be apparent to those of skill in the art that variations may be applied to the methods and it will be understood that various omissions, substitutions and changes in the form and details may be possible, without departing from the spirit of the present disclosure.

The following examples are included to demonstrate preferred embodiments of the disclosure. It should be appreciated by those of skill in the art that the techniques disclosed in the examples which follow represent techniques discovered by the inventor to function well in the practice of the invention, and thus can be considered to constitute preferred modes for its practice.

### EXAMPLE #1

In order that this aspect of the present invention is novel and inventive over the disclosure of WO 2014041440 A1, publication date Mar. 20, 2014, for the purpose of any jurisdictions in which that application constitutes prior art against the present application, in the said aspect of the present invention, the process of preparation corresponds entirely to the first three steps of preparation of such invention and may be provided in the same way and with the same embodiments, examples and preferences.

In this process, it has added the following variation in the method of manufacturing the dressing: preparing a composition including honey/additives in a preferred form, according to the first three process of preparation; and in a fourth process: combining said honey composition with a preferred elastomeric material, before gelling of the composition has initiated.

16

In this sense the way of preparing the dressing has the following sequences:

The method of manufacturing the dressing, comprising the steps:

A. add fenugreek extract (1 liter), purified honey bee 4,231 kg and Agar-Agar 72,000 g.

B. Heating for 1 hour 30 minutes (until boiling of the mixture) and mixing at 10 minutes intervals.

C. remove from heat and add 40 ml. of policresuleno (C23H24012S3 (2-hydroxy-3,5-bis[(4 hydroxy-2-methyl-5-sulfophenyl) methyl]-4-methylbenzenesulfonic Acid)

D. Strain the solution through fine mesh to avoid lumps

E. cool and keep it at 450 C

F. fill in individual trays a weight of 38.8 grams of the solution.

G. embedded in the tray the reinforcing layer of reticulated elastomeric material.

H. permit the solution/reinforcement layer to dry for 12 hours at room temperature.

I. placed and closed in appropriate packaging the final product for sterilization.

J. Product sterilization by ethylene oxide or plasma sterilization, or with γ-radiation, using a standard dose of radiation (e.g., 2.5-3.5 MRad).

Naturally, the above-mentioned steps of manufacturing devices of the present invention may further include additional steps, such as for quality control purposes. These additional steps may include visual or other types of inspections during or between the various steps, as well as final product inspection including chemical and/or physical testing and characterization steps, as well another quality control testing.

### EXAMPLE #2

### Methods of Use

The multifunctional reinforced dressing described herein may be used in both the human and veterinary fields, for temporary coverage of superficial and deep second-degree burn (partial thickness burn) and for autolytic debridement and stimulated angiogenesis in full thickness wounds, including necrotic or sloughy wounds, pressure ulcers, leg ulcers, surgical wounds, trauma wounds (abrasions, lacerations, erosions), donor sites and malodorous wounds.

Target patient group(s): patients with critically colonized wounds, patients with clinically infected wounds that are being treated with oral or intramuscular-intravenous antibiotic therapy.

### Placement Procedure for Temporary Covering:

Prepare wounds very meticulous, to guarantee lesion is entirely excised or surgically debrided, to ensure the wound bed and edges contain viable structures, free of debris and necrotic tissue.

With normal saline solution gently irrigated the injury.

Debride blister and loose skin. Cleanse with warmed normal saline.

Avoid broad area of capillary bleeding, which can lead to devitalized tissue.

Prepare/open dressing items on table. Open the outer pocket used gloved hands.

The wound dressing of the present disclosure includes a second dressing with the same material of the reinforcement structure inside the film to enhance the effect of a wound therapy treatment.

Place the product and polyethylene mesh in a sterile field.

Translated onto the wound bed and gently remove the dressing with gloved hands. Cut to size with clean or sterile scissors and apply.

The Dressing can overlap healthy tissue by 1 to 2 mm. If the wound is larger than a single dressing, then multiple dressings may be used, and if preferred, by interlacing one or more connected dressings by suturing, to cover greater extent of the wounds.

To ensure close contact, make sure careful distribution, with no wrinkles or bubbles.

Preparing Bowl with sterile saline solution and immersing the polyethylene mesh (second dressing) for 15 seconds.

The secondary dressing is applied over the primary dressing and bandaged with elastic tubular dressing (third dressing) to preserve adherence and permit easy inspections.

Dressings Fixation and compression are of significant importance to ensure the dressing adheres to the wound without shear.

Secondary dressing also stabilizes the dressing to prevent dislodgment.

In the Day, exposed the injury site to light (goose lamp 60 Watts) five minutes every two hours, for membrane desiccation for 48 hours.

Reassess wounds after 24-48 hours and see the wound. Not touch the dressing until is advantageous to remove (seven or more-day post implant).

On inspection, at 48-72 hours, the dressing changes the yellow color to a brown aspect, similar to crust.

Opaque and dry appearance indicates burn wound has re-epithelialized.

Careful inspection of the wound site and the clinical monitoring of patients' conditions allow the clinician to prolong the interval between dressing changes. It is the authors' opinion that, at most, one week of continuous without a dressing change is acceptable if no infectious signs develop.

At seven or more days the dressing may gradually peel and may be removed with gently irrigation with saline solution or by shower. They ensured pain-free removal after complete healing of the wound. Do not forcibly remove sections of the dressing that have adhere to the wound. Later, with new irrigation, they remove completely.

In some cases, it may be possible to separate the mesh and substrate by carefully pulling them apart. However, to avoid the possibility of tearing the dressing it is preferred that separation be facilitated by the use of saline solution.

Dressings to protect the practical active dressing as follows:

High-quality white polyethylene net and a Tubular Elastic Bandage applied over polyethylene layer, to protect the site and to reduce the potential of shearing and graft dislodgement.

Both can be left in place for extended periods without detrimental effects to the underlying wound. In this sense, preparing Bowl with sterile saline solution and immersing the polyethylene mesh (second dressing) for 10 seconds.

Next the wound bed is overlaid with this material in direct contact with wound surface and then bandaged with elastic tubular dressing (third dressing) to preserve adherence and permit easy inspections.

Take care in the selection of the appropriate stretch of the tubular dressing in arterial leg ulceration because they impaired circulation in this kind of patients.

Active movement are encouraged to maintain the range of motion and to reduce the amount of edema.

The use of a wound dressing in this manner has been found to promote healing by reducing the probability of infection, stimulating the deposition of granulation tissue and other beneficial processes.

Complications: The following complications are possible with the use of any wound dressings. If any of the conditions occur, the dressing should be removed: infection, inflammation, allergic reaction, excessive redness, pain or swelling. Complications that the surgeon may encounter further than infection include seroma and/or hematoma formation, and graft contracture. Although wound infection is rare, when this is suspected, appropriate bacterial identification is obligatory and according to the cultivated microorganism's antibiotics should be prescribed.

### EXAMPLE #3

### Comparative Clinical Study to Assess the Efficacy and Safety of New Multifunctional Dressing

### INTRODUCTION:

The therapeutic approach and intervention in the formation of granulation tissue in difficult to healing ulcers, suppose provide safe and quality care to people affected by these conditions.

This implies the need to investigate what may be the most effective possible protocols to guide and direct the actions of care for such chronic and complex lesions, to improve the quality of life of patients and incorporate in the country advanced and effective technology with the latest scientific knowledge and solution at a low cost.

OBJECTIVE: To compare the clinical results of treatment with traditional cures and advanced treatments with multifunctional dressing in hard-to-heal wound.

### MATERIAL AND METHODS:

The development and evaluation of a randomized trial with two treatment groups for patients with difficult to cure ulcers, one to which with a multifunctional dressing with cures every 3 days and another with wet-to-dry with gauze dressings and saline changed daily.

The study population is composed 380 adults treated in the Outpatient Department of Surgery at Roosevelt Hospital in Guatemala City in the period from January to October of 2,014, with Protocols that had been reviewed and approved by ethics committee.

Patients must meet the eligibility criteria as: both sexes, over 18 years, presence of ulceration in the lower limbs with a maximum diameter of 150 cm² and equal to or greater than 90 days of evolution.

We included eligible subjects, according to the criteria of inclusion and exclusion, with individual clarification to the patient and family about the goals and methods of the study and was provided informed consent form, which included reading, understanding and signature for inclusion in the investigation.

Treatments were performed with the multifunctional dressing in pairs new cases and in wet to dry no pairs cases, with Primary outcomes in relation to the formation of granulation tissue and/or epithelial tissue in the wound bed, with collection data measurement in 4 times (at baseline, four, eight and twelve weeks).

For the assessment of characteristics at baseline and evolution of treatment of injuries, a modified and reduced table the report of Houghton et al,¹ which included visual evaluation and measurement of five components was developed: size, type and amount of necrotic tissue, type and amount of granulation tissue.

A number from 0 to 4 was assigned to each of them, then count the five components to get the total score of the wound, with the possible total scores range from 0 to 24, with zero representing a completely healed ulcer.

Reliability, and sensitivity was determined by the indicated visual assessments and ruler measurements (maximum and maximum width of the wound length), by a single evaluator during the study period.

For the safety, were included assessment of symptoms reported by patients and clinical signs observed by the physician.

### RESULTS:

380 patients were included in the evaluation (190 in each group studied), of which 61% were female and 39% male.

Of this number only 153 patients completed the study (81%) of the multifunctional dressing group and 98 cases (52%) of the Wet-to-dry and gauze dressings with a pronounced treatment discontinuation in the first four weeks in both groups.

The greatest treatment discontinuation was particularly due to lack of patient compliance to healing, which was most evident in the treatment group Wet-to-dry and gauze dressings.

On average 3 weekly cures for the multifunctional dressing and 10 weekly cures for the Wet-todry and gauze dressings were performed.

It was determined that in 203 patients with ulcerations were diabetic patients (54%), and ulcerations due to burns, soft tissue infections in 96 cases (25%), occupying venous insufficiency third place with 54 patients (14%) and arterial disease with 27 cases (7%).

The lesions were present in the foot in 53% of cases, in the leg in 23% of them, in the sacral region in 16% of them, and trochanteric region at 8%.

Regarding the time evolution of lesions in 52% of cases ulceration was present between three to six months and the remaining 48% of cases over six months.

At the first evaluation at four weeks, it was shown that the ulcerations that were treated with the multifunctional dressing had progressed adequately to healing with intact skin or in close proximity to, in 37% of cases compared with 11% the wet dry cures.

When making the second assessment at eight weeks, it was shown that the ulcerations that were treated with the multifunctional dressing had progressed to healing with intact or near, in 50% of cases, in contrast to the progress of 20% the wet dry cures.

When performing the third evaluation at twelve weeks (end), it was shown that the ulcerations that were treated with the multifunctional dressing had progressed to healing with intact skin or in close proximity to, at 64%% of cases, in contrast to the progress of 25% of the wet dry cures.

### CONCLUSIONS

The results of a study showed that the multifunctional dressing leads to improved and accelerated tissue repair-regeneration in chronic ulcers from healing compared to healing method from the Wet-to-dry and gauze dressings.

1. A multifunctional -reinforced dressing suitable for promote or expedite the regeneration or repair of skin tissues COMPRISING: a reinforcing layer of flexible, inert, and removable reticulated elastomeric material (as polyethylene homopolymer), connected entirely to the honey/additives composition, essential to achieve the desired mechanical and physical properties for its intended function.

2. The multifunctional -reinforced dressing according to claim 1, wherein the honey/additives composition COMPRISING highly purified substances, which include in every single dressing the products and following amounts: Honey bees: 30.66 g, aqueous extract seed from Trigonella foenum-graecum 7.34 g, Policresulen 120,478 mg and gelling agent Agar-Agar 0.522 g.

3. The multifunctional-reinforced dressing according to claim 2, COMPRISING a reinforcing layer of flexible, inert, and removable reticulated elastomeric material (as polyethylene homopolymer) embedded in the honey/additives composition during gel formation, exerting a change in the appearance and physical characteristics of the dressing.

4. A method for use in Human & Veterinary Medicine, to promote or expedite the regeneration or repair of the deficient stromal connective tissue, as well as stimulating re-epithelialization, in a variety of injuries or disorders involving the skin tissues, due to trauma, diseases, or surgery, said method consist of contacting said site with the multifunctional—reinforced dressing of claims 1 to 3.

5. The process for the preparation of the multifunctional -reinforced dressing according to claim 1, COMPRISING carrying out the following steps in the order given:
A. add fenugreek extract (1 liter), purified honey bee 4,231 kg and Agar-Agar 72,000 g.
B. Heating for 1 hour 30 minutes (until boiling of the mixture) and mixing at 10 minute intervals.
C. remove from heat and add 40 ml. of policresuleno (C23H24012S3 (2-hydroxy-3,5- bis[(4-hydroxy-2-methyl-5-sulfophenyl) methyl]-4-methylbenzenesulfonic Acid)
D. Strain the solution through fine mesh to avoid lumps
E. cool and keep it at 45° C.
F. fill in individual trays a weight of 38.8 grams of the solution.
G. embedded in the tray the reinforcing layer of reticulated elastomeric material.
H. permit the solution/reinforcement layer to dry for 12 hours at room temperature.
I. placed and closed in appropriate packaging the final product for sterilization.
J. End product sterilization by ethylene oxide or plasma sterilization, or with γ-radiation, using a standard dose of radiation (e.g., 2.5-3.5 MRad).
